(19) United States
(12) Patent Application Publication
(10) Pub. No.: US 2019/0175533 A1
(43) Pub. Date: Jun. 13, 2019

(54) NUTRITIONAL SUPPLEMENT FOR IMPROVED CALCIUM ABSORPTION

(71) Applicant: Pharmco International Inc., Richardson, TX (US)

(72) Inventor: Alexander Kuo, Dallas, TX (US)

(73) Assignee: Pharmco International Inc., Richardson, TX (US)

(21) Appl. No.: 15/838,603

(22) Filed: Dec. 12, 2017

(51) Int. Cl. A61K 31/198; A61K 33/42; A61K 33/10; A61K 33/06; A61K 33/30; A61K 33/34; A61K 33/32; A61K 31/593; A61K 38/16; A23L 33/155; A23L 33/16; A23L 33/175; A23L 33/00

(52) U.S. Cl. CPC A61K 31/198; A61K 33/42; A61K 33/10; A61K 33/06; A61K 33/30; A61K 33/34; A23V 2002/00; A61K 31/593; A61K 38/168; A23L 33/155; A23L 33/16; A23L 33/175; A23L 33/30; A61K 33/32

(57) ABSTRACT

An inventive nutritional supplement for increased Ca absorption is disclosed. The inventive nutritional supplement incorporates Ca in combination with specified amounts of L-Arginine and L-Lysine. The inventive nutritional supplement may also include Copper, Zinc, Manganese and Vitamin D3 as specified herein.

### FIELD OF THE INVENTION

The present invention relates to an improved calcium supplement and, more particularly, an improved orally administered nutritional supplement that enhances the absorption of calcium in comparison to traditional supplements.

### BACKGROUND OF THE INVENTION

Calcium, the most abundant mineral in the body, is found in some foods, added to others, available as a dietary supplement, and present in some medicines (such as antacids). Calcium is required for vascular contraction and vasodilation, muscle function, nerve transmission, intracellular signaling and hormonal secretion, though less than 1% of total body calcium is needed to support these critical metabolic functions. Committee to Review Dietary Reference Intakes for Vitamin D and Calcium, Food and Nutrition Board, Institute of Medicine. Dietary Reference Intakes for Calcium and Vitamin D. Washington, D.C.: National Academy Press, 2010. Serum calcium is very tightly regulated and does not fluctuate with changes in dietary intakes; the body receives and uses calcium from bone tissue as a reservoir for, and source of calcium, to maintain constant concentrations of calcium in blood, muscle, and intercellular fluids. Id.

The remaining 99% of the body's calcium supply is stored in the bones and teeth where it supports their structure and function. Id. Bone itself undergoes continuous remodeling, with constant resorption and deposition of calcium into new bone. Whereas resorption occurs when the body takes calcium from bone tissue for critical metabolic functions and deposition occurs when the body absorbs new calcium and deposits it in the form of bone tissue. The balance between bone resorption and deposition changes with age. Bone formation exceeds resorption in periods of growth in children and adolescents, whereas in early and middle adulthood both processes are relatively equal. In aging adults, particularly among postmenopausal women, bone breakdown exceeds formation, resulting in bone loss that increases the risk of osteoporosis over time. It is vitally important to provide the body enough dietary calcium so that the body may maximize the bone resorption and deposition process and provide ample calcium reserve throughout all phases of life to prevent bone density issues in the latter stages of life. Id.

The two main forms of calcium in supplements are carbonate and citrate. Calcium carbonate is more commonly available and is both inexpensive and convenient. Due to its dependence on stomach acid for absorption, calcium carbonate is absorbed most efficiently when taken with food, whereas calcium citrate is absorbed equally well when taken with or without food. Calcium citrate is also useful for people with achlorhydria, inflammatory bowel disease, or absorption disorders. Other calcium forms in supplements or fortified foods include gluconate, lactate, and phosphate. Calcium citrate malate is a well-absorbed form of calcium found in some fortified juices.

Calcium supplements contain varying amounts of elemental calcium. For example, calcium carbonate is 40% calcium by weight, whereas calcium citrate is 21% calcium by weight. The percentage of calcium absorbed depends on the total amount of elemental calcium consumed at one time; as the amount increases, the percentage absorption decreases. Absorption is highest in doses ≤500 mg. So, for example, one who takes 1,000 mg/day of calcium from supplements might split the dose and take 500 mg at two separate times during the day. Additionally, absorption is highly dependent on the bioavailability of the form of calcium and the calcium form's solubility, hence availability for absorption, during the digestive process.

Some individuals who take calcium supplements might experience gastrointestinal side effects including gas, bloating, constipation, or a combination of these symptoms. Calcium carbonate appears to cause more of these side effects than calcium citrate due to its insolubility during the digestive process, so consideration of the form of calcium supplement is warranted. Other strategies to alleviate symptoms include spreading out the calcium dose throughout the day and/or taking the supplement with meals.

The importance of dietary calcium (Ca) intake is becoming increasingly recognized, not only for skeletal health, but also for the prevention of hypertension and colon cancer. In some industrialized countries, Ca intakes are not only often inadequate but also declining, mainly because consumption of milk and other dairy products is decreasing. At the same time prevalence of osteoporosis, a disease characterized by reduced bone mass (BMD) and micro-architectural deterioration of bone tissue, with consequent increased fracture risk, is rising. (Barclay, 2001).

During skeletal growth and maturation, i.e. until the age of the early twenties in humans Ca accumulates in the skeleton at an average of 150 mg/day until peak bone mass is reached. During maturity the body—and therefore the skeleton—is more or less in Ca equilibrium. From the age of 50 in men and from menopause in women, bone balance becomes negative and bone is lost from all skeletal sites. Due to more rapid bone loss during early menopause and lower peak bone mass, women have an increased fracture risk compared to men.

Consequently, it is of high interest for the public health to find solutions for people to reach a high peak bone mass. It is also of high interest for public health that people who have already reached their peak bone mass maintain it throughout life and minimize the risk of bone loss with age, particularly in women following the menopause.

Adequate Ca intake from the diet is critical to achieve optimal peak bone mass, and to reduce the rate of bone loss associated with aging (Flynn & Cashman, 1999). Milk and milk products are the most important dietary sources of Ca for most people in western countries, with cereal products, fruits and vegetables each making a much smaller contribution. In other nations where dairy is less of a staple, Ca intake through daily diet is not a sufficient source of dietary calcium. Additionally, only a fraction of the Ca ingested with food is absorbed and utilized by the body for metabolic functions. This is defined as the bioavailable of Ca in foods.

Ca in food occurs as salts or associated with other dietary constituents in the form of complexes of Ca ions. Ca must be released in soluble and ionized form before it can be absorbed. This makes the form of Ca and the ability of the Ca to form soluble ionized complexes vitally important to the bioavailability and availability of absorption Ca. Ca is absorbed in the intestine by two routes—transcellular and paracellular. The transcellular route involves active transport of Ca by the mucosal transport protein, calbindin and is saturable and subject to physiological and nutritional regulation via vitamin D. The paracellular route involves passive Ca transport through gap junctions between mucosal cells; it is non-saturable and essentially independent of nutritional and physiological regulation, and is concentration-dependent. Most of Ca absorption in humans occurs in the small intestine, but there is some evidence for a small colonic component.

On average, between 10 and 30% of the Ca is absorbed from a mixed diet by healthy adults. The efficiency of intestinal Ca absorption is influenced by a variety of physiological factors. Reduced efficiency in healthy individuals is observed with increasing age, menopause and vitamin D deficiency. Efficiency of absorption is increased with vitamin D excess, Ca and P deficiency and pregnancy and lactation.

Ca absorption is also influenced by a number of dietary factors. These include habitual Ca intake, Ca content of the meal, ingestion of Ca with food or without food, chemical form of Ca and gastrointestinal interaction with enhancers or inhibitors present in food and diet.

There has been considerable research effort to identify inhibitors and enhancers of Ca absorption in food with the aim to improve Ca absorption. Two well-recognized inhibitors of Ca absorption in food are oxalate and phytate. Thus, vegetables that are rich in oxalate have a low Ca bioavailability (e.g. spinach), whereas Ca absorption is higher from low-oxalate vegetable (e.g. kale and watercress). The inhibitory effect of Ca may be explained by the extremely low solubility of Ca oxalate, which makes the Ca unavailable in the intestine. Similarly it was shown that Ca absorption from high-phytate soybeans was lower than from low-phytate soybeans. The inhibitory effect of phytate may be explained by its capacity to form strong complexes with Ca in the small intestine.

A number of food constituents have been suggested as potential enhancers of Ca absorption, such as non-digestible oligosaccharides and individual milk components, e.g. lactose and casein phosphopeptides. Studies in animals showed that enhancement of Ca absorption can be achieved by addition of non-digestible oligosaccharides to the diet. These oligosaccharides are largely resistant to human digestive enzymes, and upon reaching the colon intact, they are fermented by the colonic microflora. The short chain fatty acids thus produced reduce colonic pH, resolubilizing the insoluble Ca complexes and making Ca available by passive diffusion in the colon. Several studies confirmed the enhancing effect in humans, indicating however that at moderate doses a more pronounced effect is seen only subjects which have high Ca requirements such as adolescent and postmenopausal women. Oligosaccharides for which increased Ca absorption has been shown in humans include fructooligosaccharides (FOS), galactooligosaccharides (GOS) and lactulose.

Ca is generally well absorbed from milk and diary products in humans. This has in part been explained by the positive effect of lactose and casein phosphopeptides on Ca absorption. Animal studies provide strong evidence that lactose increases Ca absorption and retention. In human infants, Ca absorption is significantly higher from soy based infant formula containing lactose than from placebo. In adults, however, lactose only has an effect on Ca absorption in β-galactosidase-deficient subjects, so that it is now concluded that there is no effect in the healthy adult population. Caseinophosphopetides (CPP) are produced in vivo and industrially by the action of proteinases on milk casein. They have been shown to possess Ca binding capacity and to maintain Ca in solution at neutral and alkaline pH. Animal studies indicated that CCP might enhance Ca bioavailability during skeletal development and prevent bone loss in older animals. Studies in humans gave somewhat inconsistent results. Whereas CPP improved Ca absorption from a standard meal no effect was found from bread meals containing low and high phytate. Similarly the addition of CCP increased Ca absorption in adults from rice-based gruel, whereas no effect was seen from a whole grain cereal. These results might indicate that the matrix of the food plays an important role and effect of CCP is restricted to foods with a moderate to low content inhibitors such as phytate.

Likewise, even fat might theoretically be viewed as an enhancer since it is known to slow gastric emptying. However, using multiple regression methods, no effect of even large variations in fat intake on absorption fraction in an observational study of middle-aged women has been found.

Nevertheless, and despite the work achieved by many scientific teams worldwide, no satisfactory solution have been proposed to resolve the problem of calcium deficiency, and only few ingredients have been established to improve Ca bioavailability from foods. Thus, there is a need for a nutritional supplement facilitating improved Ca absorption.

### SUMMARY OF THE INVENTION

Giving calcium in parallel with specific amounts of the essential amino acids L-Arginine (Arg) and L-Lysine (Lys) considerably enhances calcium absorption. Accordingly, the invention relates to the association of L-Lys and L-Arg, and nutritional product containing so. In another aspect, the invention relates to a food product having the ability to enhance calcium absorption.

Arg is an α-amino acid. It was first isolated in 1886. The L-form is one of the 20 most common natural amino acids. Arginine is a conditionally nonessential amino acid, i.e. it can be manufactured by the human body, but the biosynthetic pathway does not produce all the arginine amounts required for normal body function, such that additional amounts must be consumed through the diet. Individuals with poor nutrition or certain physical conditions typically do not have sufficient internally synthesized arginine and are advised to increase their intake of foods containing arginine. Arginine is found in a wide variety of foods, including from animal and plant sources. Arginine has been reported to play a role in the secretion of growth hormone, particularly in increasing the level of growth hormone in resting subjects taking arginine orally. However, not all studies confirmed these findings.

Lys is an α-amino acid that is used in the biosynthesis of proteins. It contains an α-amino group (which is in the protonated —NH3+ form under biological conditions), an α-carboxylic acid group (which is in the deprotonated —COO— form under biological conditions), and a side chain lysyl ((CH2)4NH2), classifying it as a charged (at physiological pH), aliphatic amino acid. It is essential in humans, meaning the body cannot synthesize it and thus it must be obtained from the diet.

Arg and Lys are involved in bone metabolism and growth. Arg is in fact involved both in the synthesis of substrates (polyamine and L-proline) implicated in collagen synthesis, and in the production of growth hormone (GH), insulin-like growth factor-I (IGF-I) and nitric oxide (NO) [1-3]. Lys effects are related to the stimulation of Ca absorption and renal conservation, and to the cross-linking process of bone collagen [4,5]. Pursuant to the invention disclosed herein, combining Arg and Lys with Ca in the ratios specified herein creates a nutritional supplement that creates increased bioavailability and, consequently, absorption of Ca.

In one embodiment of the present invention, a nutritional supplement composition for aiding the absorption of calcium to improve bone density includes 21 g-36 g Calcium per 100 g. and at least 15 g L-Arginine and L-Lysine per 100 g, wherein L-Arginine and L-Lysine are present in a two to one ratio, respectively.

In another embodiment of the present invention, the nutritional supplement composition aiding the absorption of calcium to improve bone density includes 425 mg-770 mg Zinc per 100 g, as well as 53 mg-92 mg Copper per 100 g, 107 mg-183 mg Manganese per 100 g, 260 mcg-550 mcg Vitamin D3 per 100 g, and at least 15 g L-Arginine and L-Lysine per 100 g, wherein L-Arginine and L-Lysine are present in a two to one ratio, respectively

Additional features and advantages of the present invention are described in, and will be apparent from, the following Detailed Description of the Invention.

### DETAILED DESCRIPTION OF THE INVENTION

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

According to one aspect, the present invention provides a nutritional supplement in a powder form having per 100 g powder: 21-36 g Calcium, 425-770 mg Zinc, 53-92 mg Copper, 17-183 mg Manganese, and 260-550 mcg Vitamin D3. In addition, the supplement contains 30 mg Arg and 15 mg Lys.

The following Table 1 evidences the characteristics of the supplement disclosed in this embodiment:

TABLE 1

| | | | Assay Ranges | | |
|---|---|---|---|---|---|
| Element Label Claim | | | Per 100 g | Per 1.4 g Capsule | Specification |
| Calcium | 400 | mg | 21 g-36 g | 294 mg-504 mg | 73.5%-126.0% |
| Zinc | 8 | mg | 425 mg-770mg | 5.95 mg-10.78 mg | 74.4%-134.7% |
| Copper | 1 | mg | 53 mg-92 mg | 0.742 mg-1.28 mg | 74.2%-128.0% |
| Manganese | 2 | mg | 107 mg-183 mg | 1.50 mg-2.56 mg | 75.0%-128.0% |
| Vitamin D3 (5 mcg) | 200 | IU | 260 mcg-550 mcg | 3.64 mcg-7.7 mcg | 73.0%-154.0% |
| Amino Acids* | 270 | mg | NLT 15 g | NLT 210 mg | NLT 77.8% |

| | | | |
|---|---|---|---|
| Arginine* | 30 | mg | Not Tested |
| Lysine* | 15 | mg | Not Tested |

The following Table 2 describes the inventive nutritional supplement comprising of a 1400 mg capsule for oral administration:

TABLE 2

| Description | Potency | Overage | Active/1 | | RMA/1 | |
|---|---|---|---|---|---|---|
| Calcium (Aspartate) | 13% | 3% | 2.36 | mg | 18.715 | mg |
| Calcium (Dicalcium Phosphate) | 29% | 3% | 32.34 | mg | 114.966 | mg |
| Calcium (Carbonate) | 40% | 3% | 333.96 | mg | 860.722 | mg |
| Calcium (Glycinate) | 17% | 3% | 29.49 | mg | 178.836 | mg |
| Calcium (Arginate) | 10% | 3% | 0.949 | mg | 9.784 | mg |
| Calcium (Lysinate) | 11% | 3% | 0.901 | mg | 8.444 | mg |
| Zinc (Glycinate) | 10% | 3% | 8 | mg | 82.474 | mg |
| Copper (Glycinate) | 8% | 3% | 1 | mg | 12.887 | mg |
| Manganese (Glycinate) | 18% | 3% | 2 | mg | 11.455 | mg |
| Vitamin D-3 (Cholecalciferol) | 100 IU/mg | 10% | 200 | IU | 2.222 | mg |
| L-Arginine Base | 99% | 3% | 30 | mg | 31.240 | mg |
| L-Lysine (HCl) | 80% | 3% | 15 | mg | 19.330 | mg |
| Magnesium Stearate | | | | | 15.000 | mg |
| Rice Protein Concentra | | | | | 33.925 | mg |
| | | | | | 1400.000 | mg |

| | |
|---|---|
| Total Calcium | 400 mg |
| Total Zinc | 8 mg |
| Total Copper | 1 mg |
| Total Manganese | 2 mg |
| Total Vitamin D-3 | 200 IU |

The nutritional compositions of the invention may provide minimal, partial, or total nutritional support. In preferred exemplary embodiments, the supplement is administered in conjunction with a food or other nutritional composition. In these embodiments, the compositions can either be intermixed with the food or other nutritional compositions prior to ingestion by the subject or can be administered to the subject either before or after ingestion of the food or other nutritional composition.

The supplement may, but need not, be nutritionally complete. The skilled artisan will recognize “nutritionally complete” to vary depending on a number of factors including, but not limited to, age, clinical condition, and dietary intake of the subject to whom the term is being applied. In general, “nutritionally complete” means that the nutritional supplement of the present invention provides adequate amounts of all carbohydrates, lipids, essential fatty acids, proteins, essential amino acids, conditionally essential amino acids, vitamins, minerals, and energy required for normal maintenance. As applied to nutrients, the term “essential” refers to any nutrient which cannot be synthesized by the body in amounts sufficient for normal growth and to maintain health and which therefore must be supplied by the diet. The term “conditionally essential” as applied to nutrients means that the nutrient must be supplied by the diet under conditions when adequate amounts of the precursor compound is unavailable to the body for endogenous synthesis to occur. The nutritional supplement may be provided in any form known in the art, including a powder, a suspension, a paste, a pudding, a solid, a liquid, a liquid concentrate, or a ready-to-use product. According to certain exemplary embodiment, the nutritional supplement is in a form of a powder.

The increased Ca bioavailability of the inventive nutritional supplement is demonstrated as follows:

Several hundreds of subjects were dosed using the nutritional supplements of the present invention in order to determine the effects. The following examples results are included to further describe the present invention and are not intended to limit the scope of the invention to the embodiment of the nutritional supplement in the specific examples. The example results utilize subjects ranging from fifty to seventy-five years of age. In the example results below, the subjects were prescribed the specified dosage of the nutritional supplement described in the invention.

Your bone density test results are reported using T-scores. A T-score shows how much your bone density is higher or lower than the bone density of a healthy 30-year old adult. A healthcare provider looks at the lowest T-score to diagnosis osteoporosis. Dual energy X-ray absorptiometry (DEXA) is used to determine the condition of the bone, and expressed in T value.

According to the World Health Organization (WHO): A T-score of −1.0 or above is normal bone density. Examples are 0.9, 0 and −0.9. A T-score between −1.0 and −2.5 means you have low bone density or osteopenia. Examples are T-scores of −1.1, −1.6 and −2.4. A T-score of −2.5 or below is a diagnosis of osteoporosis. Examples are T-scores of −2.6, −3.3 and −3.9. The lower a person's T-score, the lower the bone density. A T-score of −1.0 is lower than a T-score of 0.5 and a T-score of −3.5 is lower than a T-score of −3.0.

### Example 1

A sixty-five year old male subject, diagnosed with osteoporosis, took commercially available calcium supplements at the time of diagnosis. A bone scan taken initially showed that the T-score value of the subject was −2.4. After six months of taking the traditional commercially available calcium supplements, the T-score value showed that the condition of the bone density had very little improvement.

Subsequently, the subject was dosed with the nutritional supplement of the present invention, taking 2 capsules daily. Each capsule contained 1.4 g, by weight, of the nutritional supplement. In particular, each capsule contained (represented by elements): 2.36 mg Calcium (Aspartate), 32.34 mg Calcium (Dicalcium Phosphate), 333.96 mg Calcium (Carbonate), 29.49 mg Calcium (Glycinate), 0.949 mg Calcium (Arginate), 0.901 mg Calcium (Lysinate), 8 mg Zinc (Glycinate), 1 mg Copper (Glycinate), 2 mg Manganese (Glycinate), 200 IU Vitamin D3 (Cholecalcifrol), 30 mg L-Arginine Base, 15 mg L-Lysine (HCl), as well as 15.000 mg Magnesium Stearate, and 33.925 mg Rice Protein Concentrate. The Element Calcium in each capsule was 400 mg in total. Six months later, a bone scan was performed, and the T-score value had improved to −1.9. A year later, another bone scan was performed, and the T-score value had improved to −1.1. No side effects such as bloating, constipation, etc., were observed.

### Example 2

A fifty-four year old female subject was diagnosed with calcium deficiency. An initial bone scan taken showed that the score value of the subject was −1.3. Then the subject began dosage with the nutritional supplement of the present invention, taking 2 capsules daily with her meals. In particular, each capsule contained (represented by elements): 1.73 mg Calcium (Aspartate), 23.77 mg Calcium (Dicalcium Phosphate), 245.46 mg Calcium (Carbonate), 21.68 mg Calcium (Glycinate), 0.698 mg Calcium (Arginate), 0.662 mg Calcium (Lysinate), 5.95 mg Zinc (Glycinate), 0.74 mg Copper (Glycinate), 1.5 mg Manganese (Glycinate), 146IU Vitamin D3 (Cholecalcifrol), 30 mg L-Arginine Base, and 15 mg L-Lysine (HCl). The Element Calcium in each capsule was 294 mg in total. Six months later, a bone scan taken showed her T-score value improved to −0.8. No side effects such as bloating, constipation, etc., were observed.

### Example 3

A seventy-five year old female subject had injured her thoracic vertebra when she stumbled and fell. She was diagnosed with osteoporosis and a bone scan taken showed that the T-score value of the subject was −2.5. Additionally, she occasionally suffered from thoracic and lumbar pain during that time.

She was dosed with the nutritional supplement of the invention while receiving treatment for her injuries. Her dosage was two capsules daily with her meals. Each capsule was 1.8 g in weight. In particular, the capsule contains (represented by elements): 2.97 mg Calcium (Aspartate), 40.75 mg Calcium (Dicalcium Phosphate), 420.79 mg Calcium (Carbonate), 37.16 mg Calcium (Glycinate), 1.20 mg Calcium (Arginate), 1.14 mg Calcium (Lysinate), 10.78 mg Zinc (Glycinate), 1.28 mg Copper (Glycinate), 2.56 mg Manganese (Glycinate), 272 IU Vitamin D3 (Cholecalcifrol), 34 mg L-Arginine Base, and 17 mg L-Lysine (HCl). The Element Calcium in each capsule was 504 mg in total. During this period, other drugs prescribed to the subject were not excluded. After six months, a subsequent bone scan was taken showed her T-score value improved to −2.2. Additionally, the occurrence of pain for the subject was also reduced. A year later, the T-score value from the bone scan was −1.7.

### Example 4

A seventy year old male subject was diagnosed with calcium deficiency. An initial bone scan taken showed that the T-score value of the subject was −1.8. Then he began dosage of the nutritional supplement of the present invention, taking 2 capsules daily with his meals. In particular, each capsule contained (represented by elements): 2.07 mg Calcium (Aspartate), 28.30 mg Calcium (Dicalcium Phosphate), 292.22 mg Calcium (Carbonate), 25.80 mg Calcium (Glycinate), 0.83 mg Calcium (Arginate), 0.79 mg Calcium (Lysinate), 6.98 mg Zinc (Glycinate), 0.87 mg Copper (Glycinate), 1.75 mg Manganese (Glycinate), 254 IU Vitamin D3 (Cholecalcifrol), 30 mg L-Arginine Base, and 15 mg L-Lysine (HCl). The Element Calcium in each capsule was 350 mg in total. Six months later, a bone scan taken showed a T-score value of −1.3. A year later, a bone scan taken showed a T-score value of −1. No side effects such as bloating, constipation, etc., were observed.

These example results support the conclusion that the nutritional supplements of the present invention significantly increase calcium absorption and improvement of bone density. As such, the unique combination disclosed in the present invention improves absorption over typical calcium supplement.

Although particular embodiments have been described in detail herein, it is to be understood that the invention is not limited to those particular embodiments, and that various changes and modifications may be effected therein by a skilled in the art without departing from the scope or spirit of the invention as defined in the appended claims.

1. A nutritional supplement composition for aiding the absorption of calcium to improve bone density comprising of:
21 g-36 g Calcium per 100 g;
425 mg-770 mg Zinc per 100 g;
53 mg-92 mg Copper per 100 g;
107 mg-183 mg Manganese per 100 g;
260 mcg-550 mcg Vitamin D3 per 100 g; and
at least 15 g L-Arginine and L-Lysine per 100 g;
wherein L-Arginine and L-Lysine are present in a two to one ratio, respectively.

2. The nutritional supplement composition of claim 1 wherein the 21 g-36 g Calcium comprises calcium carbonate.

3. The nutritional supplement composition of claim 1 wherein the 21 g-36 g Calcium comprises calcium aspartate.

4. The nutritional supplement composition of claim 1 wherein the 21 g-36 g Calcium comprises dicalcium phosphate.

5. The nutritional supplement composition of claim 1 wherein the 21 g-36 g Calcium comprises calcium glycinate.

6. The nutritional supplement composition of claim 1 wherein the 21 g-36 g Calcium comprises calcium alginate.

7. The nutritional supplement composition of claim 1 wherein the 21 g-36 g Calcium comprises calcium lysinate.

8. The nutritional supplement composition of claim wherein the 21 g-36 g Calcium is selected from two or more of the group of calcium carbonate, calcium aspartate, dicalcium phosphate, calcium glycinate, calcium alginate, calcium lysinate.

9. The nutritional supplement composition of claim 1 in an oral dosage form selected from a tablet, a capsule, a lozenger, a chewable tablet and a bulk powder.

10. A nutritional supplement composition for aiding the absorption of calcium to improve bone density comprising of:
21 g-36 g Calcium per 100 g; and
at least 15 g L-Arginine and L-Lysine per 100 g;
wherein L-Arginine and L-Lysine are present in a two to one ratio, respectively.
